(19) United States
(12) Patent Application Publication
(10) Pub. No.: US 2006/0275806 A1
(43) Pub. Date: Dec. 7, 2006

(54) Whole genome methylation profiles via single molecule analysis

(76) Inventors: David C. Schwartz, Madison, WI (US); Gene E. Ananiev, Madison, WI (US)

Correspondence Address: QUARLES & BRADY LLP, FIRSTAR PLAZA, ONE SOUTH PINCKNEY STREET, P.O. BOX 2113 SUITE 600, MADISON, WI, 53701-2113, US

(21) Appl. No.: 11/432,112

(22) Filed: May 11, 2006

Related U.S. Application Data
(60) Provisional application No. 60/680,242, filed on May 11, 2005.
(60) Provisional application No. 60/740,583, filed on Nov. 29, 2005.
(60) Provisional application No. 60/740,693, filed on Nov. 30, 2005.

(51) Int. Cl. C12Q 1/68; C12P 19/34

(52) U.S. Cl. 435/6; 435/91.2

(57) ABSTRACT

Methods for determining methylation profiles of single polynucleotide molecules, especially genomic DNA molecules, by using sequence specificity and methylation sensitivity of restriction endonucleases and other proteins that selectively bind to methylated or unmethylated residues.

### CROSS-REFERENCE TO RELATED APPLICATIONS

This application claims the benefit of U.S. Provisional Patent Application No. 60/680,242 filed May 11, 2005; U.S. Provisional Patent Application No. 60/740,583, filed Nov. 29, 2005; and U.S. Provisional Patent Application No. 60/740,693, filed Nov. 30, 2005, each of which is incorporated herein by reference as if set forth in its entirety.

### STATEMENT REGARDING FEDERALLY SPONSORED RESEARCH OR DEVELOPMENT

This invention was made with United States government support awarded by the National Institutes of Health: DE-FC02-01ER63175 and DBI-9975606. The United States has certain rights in this invention.

### BACKGROUND

Post-replication methylation of DNA occurs most often in cytosine-guanine dinucleotides (CpGs). Methylation can be attributable to (1) de novo methylation, (2) maintenance methylation, (3) replication and methylation, and (4) replication and demethylation. About 70% of all available CpGs are methylated (mCpGs) in mammalian DNA, whereas in plants about 80% of CpGs are methylated. CpGs are underrepresented in most eukaryotic genomes because of higher mutation rates in mCpGs. By way of example, CpG mutates to TpG via deamination of carbon four of the cytosine ring. Less frequently, a guanine to adenine point mutation occurs (CpG to CpA).

Even though most CpGs in a genome are methylated, regions (“islands”) containing unmethylated CpGs are observed, usually within mammalian and plant promoter regions. Such unmethylated CpG islands are typically about 200 base pairs in length and have a guanine-cytosine content greater than 50% with about 30% of the CpGs methylated. In the human genome, 50% to 60% of all genes contain CpG islands. In plant genomes, however, about 80% of all genes contain CpG islands.

A methylation profile (i.e., presence or absence, or gain or loss, of mCpG) of a given genome varies with tissue type and represents a snap-shot of the CpGs that are modified at a given time in a given cell. A methylation profile is shown in FIG. 1A. FIG. 1B shows that the methylation profile of a mammalian genome varies throughout a lifetime. Blastocyst DNA is nearly devoid of 5-methyl cytosine. From implantation to gastrulation, however, a wave of de novo methylation occurs that establishes the characteristic lineage pattern of methylation maintained throughout the lifetime of somatic cells.

In eukaryotes, methylation plays a key role in regulating gene expression. Differential DNA methylation of maternally and paternally inherited alleles occurs in imprinting, a process that determines whether the maternal or paternal allele is expressed in a heterozygous genome. In mammals, imprinting controls embryo development; in plants, imprinting controls endosperm development. Interestingly, imprinted genes are active in mammals, but are inactive in plants.

Genetic and epigenetic DNA methylation mechanisms are also associated with cancer development and its progression. Laird P & Jaenisch R, “The role of DNA methylation in cancer genetic and epigenetics,” Annu. Rev. Genet. 30:441-464 (1996). FIG. 2 shows two epigenetic mechanisms for cancer—hypomethylation and hypermethylation. In general, hypomethylated genes have an increased potential for expression; whereas, hypermethylated genes repress transcription. Hypermethylation represses transcription via at least three non-exclusive mechanisms: (1) by altering chromatin structure, (2) by decreasing affinity of transcription factors to their cognate cis elements, and (3) by facilitating binding of methylated DNA-binding proteins (MDBPs) that block access of transcription factors to DNA. Samiec P & Goodman J, “Evaluation of methylated DNA binding protein-1 in mouse liver,” Toxicol. Sci. 49:255-262 (1999).

Generally, cancer cells exhibit a decrease in overall methylated DNA, despite an increased level of methylated DNA in CpG islands, relative to non-cancer cells. An increase in methylated DNA in CpG islands was first discovered in a human calcitonin gene. Issa J, et al., “Methylation of the estrogen receptor CpG island in lung tumors is related to the specific type of carcinogen exposure,” Cancer Res. 56:3655-3658 (1996). As noted in Table 1, additional cancer gene promoters with CpG island hypermethylation are known.

TABLE 1
Genes associated with hypermethylation.

| | Effect of Loss of Function in | |
|---|---|---|
| Gene | Tumor Development | Tumor Types |
| Rb | Loss of cell cycle control | Retinoblastoma |
| MLH1 | Increased mutation rate, drug | Colon, ovarian, endometrial and |
| | resistance | gastric tumors |
| BRCA1 | Genomic instability | Breast and ovarian tumors |
| E-CAD | Increased cell motility | Breast, gastric, lung, prostate and |
| | | colon tumors; leukemia |
| APC | Aberrant signal transduction | Breast, lung, colon, gastric |
| | | esophageal, pancreatic and |
| | | hepatocellular tumors |
| P16 | Loss of cell cycle control | Most tumor types |
| VHL | Altered protein degradation | Clear-cell renal carcinoma |
| P73 | Loss of cell cycle control | Leukemia, lymphoma and ovarian tumors |
| RASSF1A | Aberrant signal transduction | Lung, breast, ovarian, kidney and |
| | | nasopharyngeal tumors |
| P15 | Loss of cell cycle control | Leukemia, lymphoma, squamous cell |
| | | carcinoma; gastric and hepatocellular tumors |
| GSTP1 | Increased DNA damage | Prostate tumors |
| DAPK | Reduced apoptosis | Lymphoma, lung tumors |
| MGMT | Increased mutation rate | Colon, lung, brain, esophageal and gastric |
| | | tumors |

The role of genome-wide hypomethylation in cancer is not clear. One theory holds that hypomethylation leads to chromosomal aberrations. For example, mobile genetic elements (e.g., retrotransposons) are suppressed by methylation. Reactivation and subsequent movement of a mobile genetic element by hypomethylation could lead to oncogenic insertion mutations. Another theory is that hypomethylation encourages oncogene activation (e.g., H-ras and c-myc).

A large number of acute leukemias are characterized by alteration of the proto-oncogene mixed-lineage leukemia (MLL), with the most common being a removal of the C-terminus. A methyltransferase (MT) domain is located near the C-terminus and is necessary to produce functional MLL fusion proteins. In addition, the MT domain contains two copies of CGNCNNC (where N can be any nucleotide) that is responsible for binding the MLL protein to unmethylated CpGs. Birke M, et al., “The MT domain of the proto-oncoprotein MLL binds to CpG-containing DNA and discriminates against methylation,” Nucleic Acids Res. 30:958-965(2002).

Biological mechanisms for methylating DNA are a subject of great interest. In mammals, DNA methyltransferases (DNMTs) can covalently add a methyl group to carbon 5 of a cytosine ring, using S-adenosyl-L-methionine as a cofactor. DNMTs initiate methylation via a cystine-rich CXXC domain (where X can be any amino acid) that recognizes CpGs. Four mammalian DNMTs—DNMT1, DNMT2, DNMT3a and DNMT3b—have been identified. Bestor T, “The DNA methyltransferases of mammals,” Hum. Mol. Genet. 9:2395-2402 (2000); see also Hsieh C, “The de novo methylation activity of Dnmt3a is distinctly different than that of Dnmt1,” BMC Biochem. 6:6 (2005). DNMT1 is considered the primary maintenance methyltransferase (i.e., it establishes methylation patterns in daughter stands of DNA during replication). Conversely, DNMT3a and DMNT3b are considered de novo methyltransferases (i.e., they establish methylation patterns early in embryogenesis). These three enzymes may work together to establish and to maintain DNA methylation patterns. Although DNMT2 methylates DNA at a very low level, it methylates position thirty-eight in aspartic acid transfer RNA quite efficiently. Dong A, et al., “Structure of human DNMT2, an enigmatic DNA methyltransferase homolog that displays denaturant-resistant binding to DNA,” Nucleic Acids Res. 29:439-448 (2001); Goll M, et al., “Methylation of tRNA^{Asp} by the DNA methyltransferase homolog DMNT2,” Science 311:395-398 (2006).

DNMT homologs have been identified in fungi, insects and plants. For example, METI and METII of Arabidopsis thaliana are similar in structure and in function to DNMT1. Genger R, et al., “Multiple DNA methyltransferase genes in Arabidopsis thaliana,” Plant Mol. Biol. 41:269-278 (1999). The function of a third DNMT homolog remains unknown. In addition, plants have methyltransferases that are capable of methylating cytosines in the context of CpNpG (where N can be any nucleotide) and in the context of CpNpNp.

At least six methods for detecting mCpG are known. A first method uses nearest neighbor analysis, in which DNA is nick-translated with ³²P-labeled nucleotides and digested to deoxynucleoside 3′-monophosphate with a microbial nuclease. The digested DNA is applied to a thin-layer chromatography sheet and is chromatographed in two directions. Cytosine and 5-methyl cytosine appear as two distinct spots. Naveh-Many T & Cedar H, “Active gene sequences are undermethylated,” Proc. Natl. Acad. Sci. USA. 78:4246-4250 (1981), incorporated herein by reference as if set forth in its entirety.

A second method relies upon differential methylation sensitivity of restriction enzymes that recognize an identical sequence, such as HpaII and MspI. While HpaII is sensitive to methylation of an internal CpG, MspI is sensitive to methylation of an external CpG. Genomic DNA digested with either HpaII or MspI is resolved on an agarose gel. Average molecular weights of each digest are compared to determine the fraction that is not digested. Heavier methylation leads to an increased average fragment size of HpaII as compared to MspI. Bird A, “DNA methylation and the frequency of CpG in animal DNA,” Nucleic Acids Res. 8:1499-1504 (1980), incorporated herein by reference as if set forth in its entirety.

A third method uses methylation-sensitive PCR (MSP) or bisulfite PCR. Genomic DNA is denatured with NaOH and then treated with bisulfite for sixteen hours. The treatment transforms all cytosines to uracil. Following the treatment, the DNA is purified, and PCR is performed with primers designed to mimic the various methylation states. Sequencing of amplicons reveals 5-methyl cytosines as unaltered, while cytosines are uracil. Herman J, et al., “Methylation-specific PCR: a novel PCR assay for methylation status of CpG islands,” Proc. Natl. Acad. Sci. USA. 93:9821-9826 (1996), incorporated herein by reference as if set forth in its entirety. Although the MSP method is precise, its major limitation is that one needs to know the locus and its sequence. An improved MSP method, called MethyLight, uses real-time fluorescent PCR. Eads C, et al., “MethyLight: a high-throughput assay to measure DNA methylation,” Nucleic Acids Res. 28:E32 (2000), incorporated herein by reference as if set forth in its entirety.

A fourth method uses microarray technology in combination with MSP. Small oligonucleotide probes (i.e., seventeen to twenty-three nucleotides) query the status of one to four CpGs spotted onto poly-L-lysine-coated glass slides using an Affymetrix arrayer (Affymetrix; Santa Clara, Calif.). Two probes per target sequence are present on the array, one representing mCpG, and the other representing CpG. Target DNA sequences of 200 to 300 base pairs are amplified from bisulfite-treated genomic DNA using PCR. The PCR primers do not contain any CpGs, thereby making the PCR unbiased to methylation. Amplicons are labeled with either Cy3 or Cy5 dye and hybridized to the microarrays. Wei S, et al., “Methylation microarray analysis of late-stage ovarian carcinomas distinguishes progression-free survival in patients and identifies candidate epigenetic markers,” Clin. Cancer Res. 8:2246-2252 (2002), incorporated herein by reference as if set forth in its entirety.

A fifth method uses restriction landmark genome scanning (RLGS). Genomic DNA is radiolabeled at sites of rare cutting, and then is cleaved by a restriction enzyme and size-fractionated in one dimension using an agarose gel. The same DNA is further digested with a more frequently cutting restriction enzyme and size-fractionated in a second dimension. The result is multiple spots, each representing locus and copy number of a specific DNA fragment. Hatada I, et al., “A genomic scanning method for higher organisms using restriction sites as landmarks,” Proc. Natl. Acad. Sci USA. 88:9523-9527 (1991), incorporated herein by reference as if set forth in its entirety. This method has been used to study the variation in DNA methylation between different tissue types. Low throughput is a limitation of this method. In addition, the data is not given in the context of the genome and only a small number of sites are queried per assay.

A sixth method evaluates methylation status at a relatively gross level in CpG islands based upon an ability of mCpG to bind a methylation binding domain (MBD) domain. A polypeptide based on the MBD of rat methyl CpG binding protein 2 is attached to an affinity matrix and packed into a column. Because methyl CpG binding protein 2 binding to the MBD is electrostatic, it can be disrupted with salt. DNA samples run through the column are eluted using an increasing NaCl gradient. The most highly methylated DNA is found in the fraction(s) having the highest salt concentration. Shiraishi M, et al., “Methyl-CpG binding domain column chromatography as a tool for the analysis of genomic DNA methylation,” Anal. Biochem. 329:1-10 (2004), incorporated herein by reference as if set forth in its entirety. This method is limited in that it is not possible to unequivocally determine the methylation profile because DNA fragments having distinct methylation levels can exhibit similar elution profiles.

Because methylation profiles vary over a subject's lifetime, they represent a promising new clinical tool as molecular markers in pathophysiological conditions, such as cancer. Methylation profiles can be used in diagnosing, in classifying, or in monitoring a condition, even when a subject is asymptomatic. Methylation patterns can also be used in determining a prognosis. For the foregoing reasons, there is a need for improved methods in identifying and in analyzing genome-wide methylation patterns in a high-throughput manner.

### BRIEF SUMMARY

The present invention relates to methods for characterizing regions of a polynucleotide at the nucleotide sequence level as to hypomethylation or hypermethylation status (hereinafter, a “methylation profile”) and to systems for practicing the methods. The methods and systems employ optical polynucleotide mapping techniques, in silico digestion analysis using known polynucleotide sequences to identify the fragment(s) being mapped, as well as methods for identifying methylation status of particular sites, the location(s) of which can be mapped to the fragment(s) identified by optical mapping. Methylation sites can be identified, for example, either by cleaving polynucleotides with sequence-specific restriction endonucleases having defined sensitivities to methylation in a polynucleotide. Alternatively, methylation site-specific reagents, such as proteins, polypeptides or protein domains having known ability to selectively bind to methylated or to unmethylated residues in nucleic acid, can be labeled and visualized in conjunction with optical mapping to identify the position on a mapped polynucleotide of the binding site, which can be correlated with relevant sequence information. In some embodiments, the methylation site-specific reagent is a protein having a domain that binds a methylated polynucleotide, such as methylated DNA binding protein 1, methylated DNA binding protein 2, methylated DNA binding protein 3, methyl-CpG binding protein 1 or methyl-CpG binding protein 2.

In one aspect, the invention is summarized in that a method for establishing a methylation profile of an elongated, immobilized, sequence-characterized polynucleotide includes the steps of preparing sequential optical maps depicting sites at which the polynucleotide is cleaved by restriction enzymes. The first restriction enzyme is typically a methylation-insensitive restriction enzyme that cleaves methylated restriction sites or that cleaves restriction sites that lack methylation; whereas, the second restriction enzyme is a methylation-sensitive restriction enzyme that does not cleave methylated restriction sites; and comparing the optical maps to establish the methylation profile of the polynucleotide. From the methylation profile one can identify regions of hypomethylation or hypermethylation in the polynucleotide. To aid in identifying regions of hypomethylation or hypermethylation, an in silico barcode is constructed for each optical map prior to alignment. The barcode serves as a convenient means to compare data with available annotations regarding genes, regulatory regions and expression.

In some embodiments, the methylation-sensitive restriction enzyme and the methylation-insensitive restriction enzyme are added simultaneously to generate a single optical map. In other embodiments, the enzymes are added sequentially.

In some embodiments, the polynucleotide is an isolated genomic DNA molecule which, when isolated from a cellular or tissue source, retains the characteristic methylation profile of the source. In other embodiments, the polynucleotide is any other polynucleotide that includes methylated nucleotides, without regard to whether the nucleotides are methylated in vivo or in vitro.

In another aspect, the present invention is summarized in that methods of diagnosing, of classifying or of monitoring in a subject a status of a condition affected by methylation of a polynucleotide include the step of comparing a methylation profile of the polynucleotide to a methylation profile of the polynucleotide obtained from a subject having a predetermined status of the condition.

In certain embodiments, the polynuclucleotide is obtained from the subject before diagnosis of the condition, before a treatment for ameliorating the condition or after a treatment for ameliorating the condition. In some embodiments, monitoring of the methylation profile over a time course can assist in assessing disease progression. In some embodiments, the condition is a cancer.

In another aspect, the invention is summarized in that an apparatus for carrying out the optical mapping aspects of the methods of the invention includes a polynucleotide immobilizing device, a polynucleotide imaging device and image analysis system.

The previously described embodiments of the present invention have many advantages, including a first advantage that large quantities of genomic DNA can be screened in a high-throughput manner and that polynucleotide methylation profiles can be assigned to defined genomic loci where the genomic map of the subject is known at the molecular level.

Another advantage is that methylation profiles can be obtained without chemical conversion of native cytosine to another base (e.g., uracil) and without hybridization steps.

These and other features, aspects and advantages of the present invention will become better understood from the description of preferred embodiments that follows. In the description, reference is made to the accompanying drawings, which form a part hereof and in which there is shown by way of illustration, not limitation, embodiments of the invention. The description is not intended to limit the invention to cover all modifications, equivalents and alternatives. Reference should therefore be made to the claims recited herein for interpreting the scope of the invention.

### BRIEF DESCRIPTION OF THE DRAWINGS

The invention will be better understood and features, aspects and advantages other than those set forth above will become apparent when consideration is given to the following detailed description thereof. Such detailed description makes reference to the following drawings, wherein:

FIG. 1A shows (1) de novo methylation, (2) maintenance methylation, (3) replication and methylation, and (4) replication and demethylation; FIG. 1B shows the time course of the formation of a methylation profile during development;

FIG. 2 shows genetic and epigenetic mechanisms of DNA methylation in cancer;

FIG. 3A shows an image of the restricted, single genomic DNA molecule; FIG. 3B shows the negative of FIG. 3A aligned with a representation of the restriction fragments of the molecule (called a “bar code”);

FIG. 4A shows a single genomic DNA molecule having 5-methyl cytosines within the restriction sites of the chosen restriction endonuclease (“overlapping methylation”) and outside of the restriction sites (“nonoverlapping methylation”); FIG. 4B shows the expected bar code representation of the restriction fragments of the molecule of FIG. 4A; FIG. 4C shows the methylation map of the optical mapping bar code;

FIG. 5A shows an alignment of empirical genomic bar codes from a methylation-sensitive restriction enzyme to an in silico genome map using a pairwise comparison program—SOMA; FIG. 5B shows an alignment of a restriction map assembled de novo as compared to an in silico map;

FIG. 6A-D show bar codes generated by multiple restriction digestion to generate fine-scale methylation maps of a nucleic acid molecule.

FIG. 7A shows alignment of at least one fluorochrome-labeled methylation binding domain (MBD) aligned to a bar code of a digested, single genomic DNA molecule having methylation sites and the in silico digest of the genomic sequence showing the sequence recognized by the MBD; FIG. 7B shows DNA binding lifetime of methylated DNA binding protein 2 (MDBP2); FIG. 7C shows DNA binding lifetime of the MT domain of the MLL protein;

FIG. 8 shows the use of fluorochrome-labeled protein fragments having a methylation binding domain to identify mCpG.

FIG. 9 depicts in schematic fashion sequential (A) and simultaneous (B) cleavage of a polynucleotide corresponding to an in silico digest (bar code).

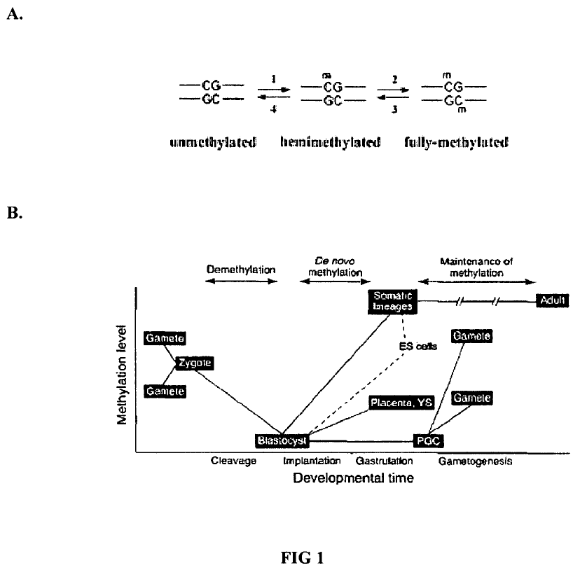

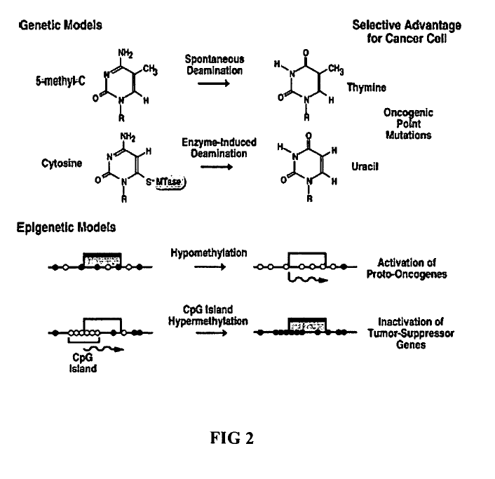

### DESCRIPTION OF PREFERRED EMBODIMENTS

Unless defined otherwise, all technical and scientific terms used herein have the same meaning as commonly understood by one of ordinary skill in the art to which the invention belongs. Although any methods and materials similar to or equivalent to those described herein can be used in the practice or testing of the present invention, the preferred methods and materials are now described.

BisI, a methylation-insensitive restriction endonuclease (i.e., that cleaves mCpG either because mCpG does not block its recognition site or because recognition site lacks mCpG) was described by Chmuzh, E. et al., “Restriction endonuclease BisI from Bacillus subtilis T30 recognizes methylated sequence 5′-G(m5C) NGC-3′,” Biotechnologia 3:22-26 (2005). BisI is a second member (after DpnI, which recognizes a methylated adenine) of subtype IIM enzymes, which cleave only methylated DNA. Other methylation-insensitive restriction endonuleases include BamHI or SwaI. Suitable methylation-sensitive (i.e., will not cleave mCpG because mCpG blocks recognition site) restriction endonucleases can be EagI, NheI, NotI, StuI or XhoI. The present invention, however, is not intended to be limited to particular restriction enzymes, as a skilled artisan is familiar with other methylation-insensitive and methylation-sensitive restriction enzymes. A particularly useful resource for determining whether a given restriction enzyme is methylation-insensitive or methylation-sensitive is The Restriction Enzyme Database (REBASE®), available on the world-wide-web. Roberts R, et al., “REBASE—restriction enzymes and DNA methyltransferases,” Nucleic Acids Res. 33(Database issue):D230-232 (2005), incorporated herein by reference as if set forth in its entirety.

Other suitable methylation-specific reagents that can be labeled and visualized at positions of hypermethylation or hypomethylation include, but are not limited to, methylated DNA binding protein 1 (MDBP-1), methylated DNA binding protein 2 (MDBP2), methylated DNA binding protein 3 (MDBP-3), methyl-CpG binding protein 1 (MeCP1) and methyl-CpG binding protein 2 (MeCP2), as well as fragments thereof that retain the ability to bind to methylated nucleotide sites.

Optical Mapping (OM) is a technique for creating physical maps of individual DNA molecules based on ordered polynucleotide restriction enzyme maps, and advantageously for creating physical maps of whole genomic DNA based on ordered maps of individual genomic DNA molecules. OM is a versatile platform in genomics research for constructing physical maps of multiple genomes, for discovering new genome structures, for facilitating sequence assembly and for comparing microbial genomes. Aspects of OM are disclosed in U.S. Pat. Nos. 5,405,519; 5,599,664; 5,720,928; 6,147,198; 6,150,089; 6,174,671; 6,221,592; 6,294,136; 6,340,567; 6,448,012; 6,509,158; 6,610,256 and 6,713,263; each of which is incorporated herein by reference as if set forth in its entirety. Likewise, additional aspects of OM are disclosed in US Patent Application Nos. 2003/0036067; 2003/0087280; 2003/0124611 and 2005/0234656; each of which is incorporated herein by reference as if set forth in its entirety.

When genomic DNA molecules are optically mapped, methylation patterns are preserved, whereas these patterns are absent from clones in constructed libraries and from PCR amplification products. Because maps of imaged restriction endonuclease fragments of single molecules (“bar codes”) can be indexed to known sequence data (e.g., NCBI Build 36 of the human genome sequence; available through the National Center for Biotechnology Information), anonymous genomic DNA molecules are confidently identified and linked to annotation at the level of the single nucleotide. The present invention leverages this bar coding scheme to further identify methylation sites on such characterized molecules. Given the high throughput of this OM system, an entire genome can be rapidly scanned to reveal novel methylation patterns.

Suitable fully integrated OM systems for use with the present invention have been previously described. Such systems incorporate microfluidics, modalities for molecular interrogation, operator-free image acquisition, machine vision, molecule-to-map analysis, aligning software, database structures for all operations and a myriad of user interfaces for data acquisition and visualization.

In an OM system, high-molecular weight molecules, such as genomic DNA, are immobilized using a microfluidics device. The genomic DNA molecules can be affixed to an OM surface or can be immobilized and elongated in nano-dimensional channels without being affixed thereto. The nano-dimensional channels can, for example, have a height on the order of 30 nm effectively constraining the DNA from substantial curvature in a vertical plane based on the cross-sectional diameter of the DNA. This constraint ensures that the DNA is maintained within the focal plane of a microscope objective which may view the DNA through a transparent top wall of the nanochannels. The width of the channel may be on the order of 1,000 nanometers or one micrometer. This allows for simple fabrication of the channel using elastomeric molding techniques, for example, and improves the ability to draw the DNA into the nanochannels. The increased stiffness of the DNA preserves its orientation and alignment in the nanochannels despite the width of the nanochannels.

The DNA may then be optically mapped or manipulated in other ways within the nanochannels. One wall of the nanochannels may be semi-permeable to allow additional chemical reactions to take place affecting the DNA, for example, restoring salt to the DNA. When employing such an immobilization strategy that does not entail attachment to or interaction with a surface, DNA in solution can include a number of nicks in which one strand is broken. Such nicks may result from damage from mechanical damage, UV light, or temperature. These incidental nicks are repaired to prevent confusion with nicks made for marking purposes. The suspended DNA, as repaired, may be nicked at predefined base pair sequences by enzyme. These nicks, when identified in position, will reveal important properties of the DNA. Salt can be removed from the DNA by adjusting its buffering solution to cause an elongation of the DNA and a corresponding increased rigidity. Nicks produced by enzyme can be labeled with a fluorochrome providing a given frequency of light emission, and the remaining body of the DNA may be labeled with a second fluorochrome having a second frequency of emission. The first fluorochrome may be uniquely keyed to the point of the nick whereas the second fluorochrome may distribute themselves uniformly over the remaining surface of the DNA. The DNA may thus be imaged using Fluorescence Resonance Energy Transfer. The elongated and labeled DNA in solution may then be removed, for example by a pipette, and transferred into a first chamber of a nanochannel assembly. The first chamber provides one electrode of an electrophoresis device and communicates through a set of nanochannels with a second chamber of the assembly having a second electrode of the electrophoresis device. As will be understood in the art, operation of a voltage across the electrodes will draw charged molecules such as DNA from the first chamber to the second chamber through the nanochannels extending therebetween. This step entraps the DNA within the nanochannels for analyses or subsequent processing.

The immobilized DNA molecules are cleaved by digestion with a restriction enzyme; and contiguous, immobilized cleavage fragments are imaged and sized by, e.g., fluorescence microscopy. The imaging can be performed automatically by commercially available machine vision software, i.e., Pathfinder, whose output is large map files. Pathfinder also determines the fragment mass measurements. These map files are overlapped to construct whole-genome maps with the map assembler and viewed by Genspect, which displays aligned maps, linked annotations and presents the user with a variety of editing tools and analysis. See Dimalanta E, et al., “A microfluidic system for large DNA molecule arrays,” Anal. Chem. 76:5293-5301 (2004); and Zhou S, et al., “A single molecule system for whole genome analysis,” in New Methods for DNA Sequencing (Mitchelson K, ed. in press), each of which is incorporated herein by reference as if set forth in its entirety. Genome Zephyr, a new imagining system, can acquire and process 2,000 images/hour or 60,000 images in about 30 hours, corresponding to an approximate 4-fold coverage of the human genome. Id. Omarie, an interactive image viewer, enables the user to rapidly browse and to interact with large superimages consisting of hundreds of overlapped digital micrographs showing genomic DNA molecules. Id.

The OM system creates and reads hundreds of thousands of single molecule restriction maps, producing a representation of the imaged restriction fragments known as bar codes. Bar codes are aligned to fragments predicted by in silico digests of known nucleic acid sequence and assembled in displays for comparison. Multiple displays are aligned and are assembled to form a contig map that can be compared to the known sequence.

One embodiment of an OM system is described in greater detail below. See generally, Zhou S, et al., “Single-molecule approach to bacterial genomic comparisons via optical mapping,” J. Bacteriol. 186:7773-7782 (2004); Zhou S, et al., “Whole-genome shotgun optical mapping of Rhodobacter sphaeroides strain 2.4.1 and its use for whole-genome shotgun sequence assembly,” Genome Res. 13:2142-2151 (2003).

Sample Preparation: In one embodiment, glass coverslips (22 mm by 22 mm—Fisher's Finest; Fisher Scientific; Pittsburgh, Pa.) are racked in custom-made Teflon racks, cleaned by boiling in Nano-Strip (sulfuric acid and hydrogen peroxide; Cyantek Corp.; Fremont, Calif.) for 50 minutes at 68° C. to 75° C., and then rinsed extensively with high-purity, dust-free water. After six washes, the surfaces are hydrolyzed in boiling concentrated hydrochloric acid at 98° C. for 6 hours and rinsed extensively with high-purity water until the wash is neutral. The coverslips are removed from the Teflon racks and individually rinsed three times in absolute ethanol. They are then stored in absolute ethanol in polypropylene containers at room temperature. Using forceps, the cleaned, hydrolyzed cover slips are placed in a flat Teflon block holder in a clean Qorpak container and allowed to dry for five to ten minutes at room temperature. High-purity water (250 ml) is added to a clean polypropylene bottle, to which 62 μl of trimethyl silane (N-trimethylsilylpropyl-N,N,N-trimethylammonium chloride; Gelest Corp.; Tullytown, Pa.) and 3 μl of vinyl silane (vinyltrimethoxysilane; Gelest Corp.) are added and shaken vigorously for several minutes. The solution is poured into the Qorpak container and incubated at 65° C. with gentle shaking (50 rpm) for 17.5 hours. The container is then opened in a hood for one hour to thermally equilibrate. Finally, the silane solution is aspirated off and the resulting derivatized surfaces are rinsed three times with high-purity water and once with ethanol and then stored in distilled absolute ethanol. The derivatized surfaces remain usable for two to three weeks. Surface properties are assayed by digesting lambda DASH II bacteriophage DNA with a chosen restriction enzyme, such as 40 units of XhoI (methylation-sensitive) diluted in 100 μl of digestion buffer with 0.02% Triton X-100 (Sigma-Aldrich; St. Louis, Mo.) at 37° C. or 40 units of BamHI (methylation-insensitive) diluted in 100 μl of digestion buffer with 0.02% Triton X-100 (Sigma-Aldrich) at 37° C. to determine optimal digestion times, which ranged from 40 minutes to 120 minutes.

DNA Mounting, Overlaying, Digesting and Staining: DNA molecules can be mounted on a derivatized glass surface using a microfluidic device. See Dimalanta et al., supra. Then, a thin layer of acrylamide (3.3%; 29 parts acrylamide to 1 part N,N′-methylene-bisacrylamide, 0.075% ammonium persulfate, 0.1% tetramethylethylenediamine and 0.02% Triton X-100) is applied to the surface, which upon polymerization is washed with 400 μl of TE for 2 minutes, followed by washing with 200 μl of digestion buffer for another 2 minutes. To set up the digestion, 200 μl of digestion buffer containing enzymes (20 μl of NEB; New England Biolabs; Ipswich, Mass.), 10× buffer 2, 176 μl high purity water, 2 μl of 2% Triton X-100 and 2 μl of NEB-BamHI (20 U/μl)) is added to the surface, followed by incubation in a humidified chamber at 37° C. for 40 minutes to 120 minutes. After digestion, the surface is washed twice with 400 μl of TE for 2 minutes to 5 minutes and the TE is aspirated from the surface. The surface is mounted onto a glass slide with 12 μl of 0.2 μM nucleic acid dye 1,1′-[1,3-propanediylbis[(dimethyliminio)-3,1-propanediyl]]bis[4-[(3-methyl-2(3H)-benzoxazolylidene)methyl]]-tetraiodide (YOYO-1) solution (containing 5 parts of YOYO-1 and 95 parts β-mercaptoethanol in 20% (vol/vol) TE). The sample is sealed with nail polish and incubated at 4° C. (in the dark) for 20 minutes or overnight for the staining dye to diffuse before checking by fluorescence microscopy.

Image Acquisition and Processing: DNA samples can be imaged by fluorescence microscopy with a 63× objective lens (Zeiss; Oberkochen, Germany) and a high-resolution digital camera (Princeton Instruments; Trenton, N.J.). ChannelCollect collects images by using a fully automated image acquisition system developed for this purpose. See Dimalanta et al., supra; and Zhou et al. (in press), supra.

Co-mounted lambda DASH II DNA molecules are used to estimate the digestion rate and to provide internal fluorescence standards for accurately sizing the DNA fragments. The image files are processed as described supra to create maps.

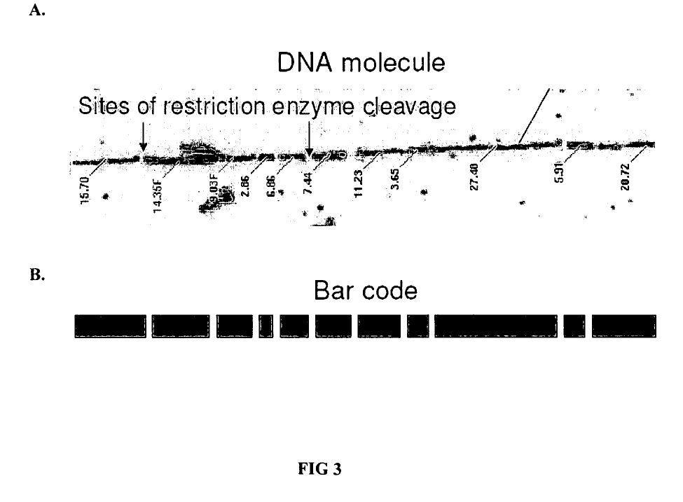

FIG. 3A is a negative image of a restricted single genomic DNA molecule, aligned with a representation of the restriction fragments of the molecule, called a bar code, shown diagrammatically in FIG. 3B. Because an internal standard of known sequence and size is used, sizes of identified restriction fragments can be assigned to individual fragments. Because the fragments are immobilized, the fragments maintain their native order relative to one another such that a bar code showing both the relative position and size of individual fragments of a larger polynucleotide can be generated.

Optical Map Assembly: Individual molecule restriction maps are overlapped by dedicated optical map assembler software. Briefly, the software assembles single molecule restriction maps into a genome-wide map contig using a computationally efficient algorithm with limited backtracking for finding an almost optimal scoring set of map contigs to avoid the high computational complexity that would occur in attempting to find the optimal assembly. Bayesian inference techniques are used to estimate the probability that two distinct single molecule restriction maps could have been derived from the proposed placement while subject to various data errors such as sizing errors. The Bayesian inference approach requires the fine-tuning of these parameters and a known prior statistical distribution of error sources. Important measures of data quality, such as measurement standard deviations, digestion rate, false cut and false match probability can be estimated from the data by using a limited number of iterations of Bayesian probability density maximization. After these parameters are correctly estimated from the data, a dynamic programming algorithm computes a best offset and alignment between a pair of maps.

Map Homologies: Map homologies are scored by first using a sliding window to break a whole-genome restriction map (optical map or in silico map) into “segments” consisting of ten consecutive restriction fragments, at two fragment intervals. This scoring produces a series of overlapping map segments, which are aligned pairwise and merged (with other alignments) using a modified version of the map assembler, against a second reference map constructed from a mapped or sequence genome. Since the map assembler performs global alignments, only highly congruent maps were aligned. Differences stemming from fragment sizing errors and missing or spurious cut sites have been previously modeled and accounted for within the assembly software. However, gross local map differences are not accounted for in this alignment process and are partly compensated for by the alignment of relatively small maps against the reference. Resulting alignments are merged into single consensus maps for comparison against the reference map. The merging process produces a single consensus map in much the same way single-molecule maps are combined to create a whole-genome map. As such, some regions of homology across a given pair of strains may not have been accounted for. Given these caveats, we estimated the percentage of genome homology by simply summing the fragment sizes of homologous regions, defined as only regions covered by the previously described alignment and merging process.

Coding versus non-coding restriction enzyme cleavage sites are tabulated by comparing the nucleotide coordinates of the given enzyme recognition sites in the genome sequence with the coordinate ranges for the genes in a genome sequence annotation. If the coordinates for any given restriction site is within the coordinate range of any given gene, this restriction site was considered within a coding region. All other restriction sites are scored as residing within non-coding regions.

Annotation: Variant loci are detected by comparing maps that are characterized using annotation-derived, sequenced Shigella flexneri strains. Basically, the coordinate ranges for the fragments, which vary among these strains, are guided by whole-genome in silico maps. Corresponding sequences are aligned at the nucleotide level using MegAlign (DNAStar; Madison, Wis.) to recognize insertion or deletions between the two sequenced strains and annotations from the National Center for Biotechnology Information (NC_{—}004337 and NC_{—}004741).

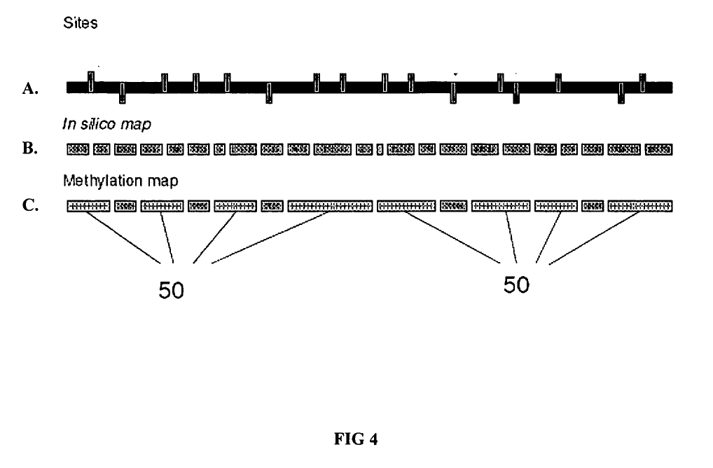

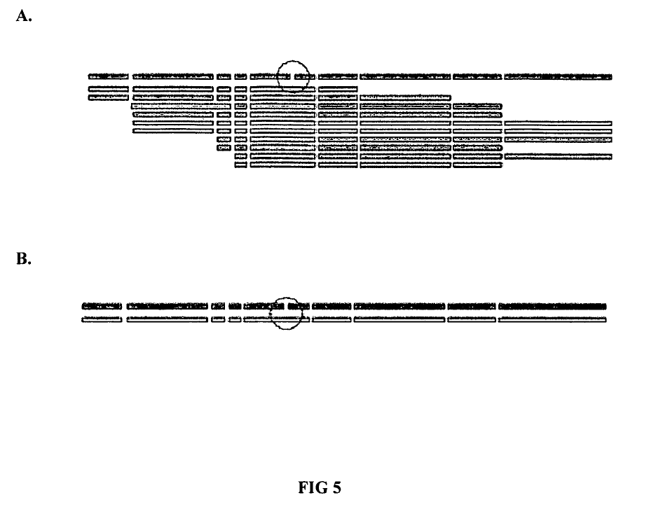

FIG. 4A-C depict a methylation profile of a single genomic DNA molecule having a known nucleic acid sequence, but an unknown methylation profile. The DNA molecule of FIG. 4A includes 5-methyl cytosine residues both in the cleavage/recognition sites of the restriction enzyme (a condition referred to as “overlapping methylation”) as well as outside the sites (“nonoverlapping methylation”). The profile is established by digesting the DNA molecule, in vitro and in silico, with a restriction enzyme that cannot cleave the molecule when a cytosine residue in its recognition/cleavage site is methylated (i.e., is 5-methyl cytosine). FIG. 4B depicts in bar code format the restriction fragments predicted by digestion of the polynucleotide in silico. FIG. 4C shows that the restriction enzyme fails to cleave the nucleic acid at all of the predicted sites, resulting in longer than predicted fragments which indicate the presence of 5-methyl cytosine residues (i.e., methylated DNA) at some of the predicted cleavage sites.

FIG. 5A-B show two approaches for studying the methylation profiles of genomic DNA using restriction endonucleases that will not cleave at sites containing mCpG. FIG. 5A shows an alignment of empirical genomic bar codes to an in silico genome map using the pairwise comparison program, SOMA, which reveals a site of CpG methylation as a cut that is missing (indicated in the circle) in the genomic DNA bar codes. FIG. 5B shows an alignment of a restriction map assembled de novo that is then compared to an in silico map, which also reveals a site of CpG methylation as a cut that is missing (as indicated in the circle) in the de novo restriction map.

### EXAMPLES

### Example 1

### Optical Mapping with Methylation-Sensitive Restriction Endonucleases

A. thaliana was studied as a model system for the following reasons: (1) the A. thaliana genome is small, 120 MB; (2) the A. thaliana genome is sequenced and annotated; (3) the A. thaliana genome contains a total of 2,786,890 CpGs; (4) the A. thaliana genome has cytosine methylation at both CpG and CpNpG sites; and (5) the A. thaliana genome has a relatively low level of genomic repeats when compared to genome's of other organisms.

A T87 cell line of A. thaliana was grown in the presence of 2,4-dichlorophenoxyacetic acid (2,4-D). 2,4-D induces not only a change in the morphology of the cell, but also a change in the methylation profile of the cell. The T87 cell line was initiated in 1992 from the Columbia ecotype of A. thaliana—the same ecotype that has been sequenced.

A DNA isolation protocol solves the problems of chlorophyll contamination of DNA, of cell wall debris and of starch granules. The DNA isolation protocol is adapted from a procedure for the preparation of nuclei, similar to a fiber fish protocol. Weier H, “DNA fiber mapping techniques for the assembly of high-resolution physical maps,” J. Histochem. Cytochem. 49:939-948 (2001). Briefly, tissue samples are ground in liquid nitrogen and placed in a sucrose buffer. The resulting solution is filtered, and the chloroplasts are lysed using Triton X-100 (0.5% final concentration). Nuclei are isolated by centrifugation and then lysed in NDSK (0.5 M EDTA, pH 9.5, 2% N-lauroyl-sarcosine and 2 mg/ml proteinase K). Consequently, DNA-bound proteins are largely removed.

Bar codes generated with a methylation-sensitive restriction enzyme, XhoI, were used for creating a restriction map without the use of any prior sequence knowledge. This de novo restriction map was then compared to the in silico map of the genome to determine sites of methylation by cataloging differences between the two maps.

The studies were performed as follows: A. thaliana genomic DNA was mixed with E. coli genomic DNA in a 10:1 ratio. DNA from bacteriophage Lambda DASH II was added to the genomic DNA mixture to a final concentration of 15 pm/μl to act as an external size standard. E. coli DNA does not contain any CpG methylation—it was used as an internal control to assay the digestion rate. The methylation-sensitive restriction enzyme XhoI, capable of discerning the methylation state of 17,388 CpG, was used for optical mapping. The data set generated representing 412× coverage of the A. thaliana genome and is summarized below in Table 2.

TABLE 2
Summary of the T87 XhoI Data Set.

| | A. thaliana | E. coli |
|---|---|---|
| Number of Molecules | 117,922 | 9,857 |
| Average Molecule Size | 419 kb | 478 kb |
| Expected Average Fragment | 6.86 kb | 26.06 kb |
| Size | | |
| Observed Average Fragment | 18.76 kb | 25.29 kb |
| Size | | |

SOMA was used to separate E. coli bar codes from A. thaliana bar codes. SOMA performs a pair-wise alignment of individual molecular bar codes to the in silico restriction map of a genome. A molecule is placed at a specific location in the genome and a numerical score is assigned to indicate the quality of the match. Higher numerical scores represent better quality alignments. A numerical score of 5 was used for E. coli molecules. For A. thaliana, the numerical score used was 3.5—the default level stringency.

If DNA methylation in the A. thaliana T87 cells was clonal, one would expect to see a cut missing from a significant portion of the bar codes aligned to a genomic locus in SOMA alignments. However, the missing cuts were not uniformly distributed, indicating that DNA methylation in T87 cells is aclonal or heterogeneous. We further tested the clonality of this data set (bar codes aligned by SOMA) by attempting to assemble the map piles (bar codes that aligned to the in silico map) into a composite optical map. The assembly yielded 257 contigs, with the largest one being 1.9 MP long. The aclonality of T87 cells is consistent with data from maize plant cell cultures.

The level of CpG methylation in XhoI restriction sites in T87 cells is about 70%, which is consistent with the general notion of methylation levels of non-repeat based CpG. The percentage of methylation sites was derived from comparing expected (6.86 kb) versus observed (18.76 kb) average fragment sizes.

The assembly of a de novo physical map was limited by the large size of the data set. The initial data set was broken up into 10,000 map cluster and analysis was performed using medium stringency parameters on each of the sets. This approach produced 640 contigs ranging in size from 1.8 mb to 350 kb.

The de novo E. coli K12 map verified a digest rate of close to 90%. Bar codes over 1 MB in size matching E. coli were assembled into a complete physical map using map assembler software. Filtering was necessary to shrink the size of the map data set. The complete E. coli map data set represents approximately one-thousand-fold coverage of the E. coli genome. The time needed to assemble a map with this level of coverage would be very long. The filtered data set contained 95 molecules representing a 25× coverage. Medium stringency parameters were used for the map assembly. The resulting optical map was of good quality as indicated by a false circular probability score of 0.0048. The map indicated a digest rate of 87.5% and a false cut rate of 0.7% in the DNA molecules contained in the contig.

### Example 2

### Detecting Endogenous and Exogenous Methylation in a Genome

AluI methylase methylates cytosine in AGCT, while NheI cleaves DNA at GCTAGC. AluI methylation thus produces an overlap of NheI cleavage sites at 5′-AGCTAGC-3′ and 3′-GCTAGCT-5′. The E. coli genome contains 158 NheI cleavage sites. NheI cleavage is blocked by cytosine methylation and, therefore, longer restriction fragments are generated when NheI digests methylated, as opposed to unmethylated DNA.

E. coli DNA molecules were methylated de novo using AluI methylase and then were cleaved with NheI. A circular contig map of the resulting fragments generated from an optical mapping display of 176 resulting maps (twenty-fold coverage of the E. coli genome) was compared to an in silico NheI map of the E. coli genome. This model was used to avoid issues with non-clonality and to assess errors.

Dcm methylation of the inner cytosine in a CCWTT sequence occurs naturally in wild-type E. coli, including the MG1655 strain, as a defense mechanism. As with NheI, methylation blocks cleavage by StuI. The E. coli genome contains 606 StuI cleavage sites, 147 of which are blocked by Dcm methylation, and of those, 74 sites are blocked by adjacent methylation.

Dcm-methylated E. coli DNA molecules were isolated and cleaved with StuI. A circular contig map of the resulting fragments generated from an optical mapping display of 469 resulting maps (fifty-fold coverage of the E. coli genome) was compared to an in silico StuI map of the E. coli genome. 144 methylation sites were identified as missing cuts, and three expected sites could not be identified. Like AluI-methylation, naturally occurring E. coli Dcm-methylation was used to avoid issues with non-clonality and to assess errors.

### Example 3

### Using Sequential and Simultaneous Multiple Restriction Endonuclease Digests in Optical Mapping of Methylation

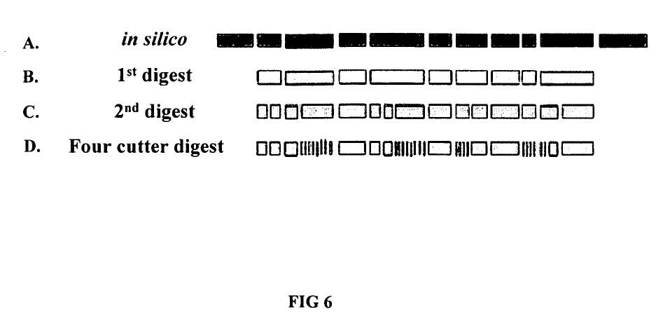

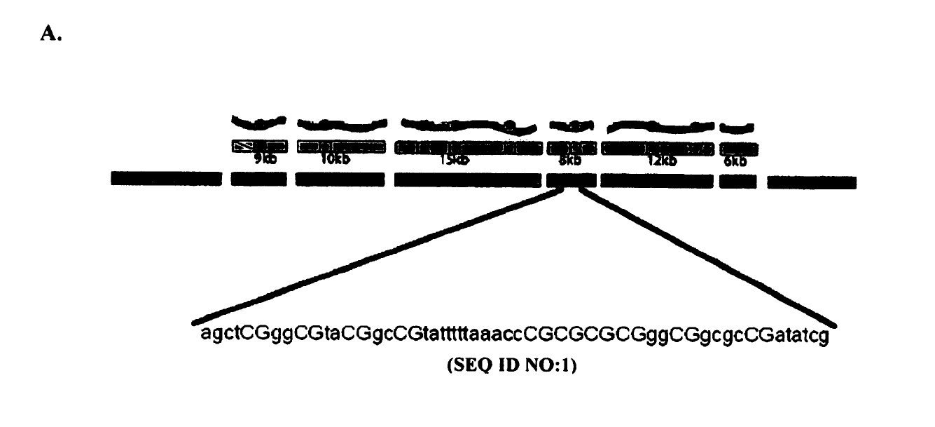

For fine-scale methylation mapping, it can be advantageous to perform multiple restriction enzyme digests on the same isolated single genomic DNA molecules. The optical mapping system permits the re-identification of the same molecule after multiple digest. For example, one can first derive basic bar codes to index the DNA molecules using a first restriction enzyme that is unaffected by the state of CpG methylation, followed by a second digest using a methylation-sensitive restriction endonuclease to explore the methylation profile. FIG. 6A-D illustrate how to identify methylation sites using bar codes that result from multiple restriction digests. In FIG. 6A, the bar code expected from an optical mapping-based in silico digest of a single genomic DNA molecule with a first restriction endonuclease. FIG. 6B, C and D, align experimentally-derived bar codes produced by an in vitro restriction digest with the first restriction endonuclease, with the second restriction endonuclease, and with a third restriction endonuclease.

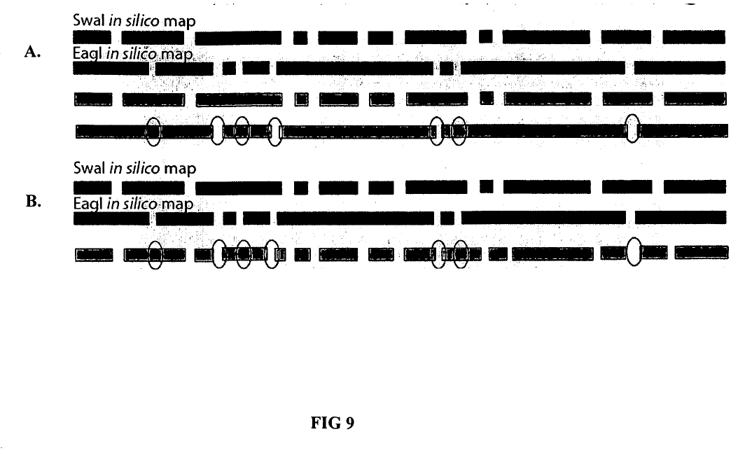

In a separate experiment, depicted in FIG. 9, detection of CpG methylation was achieved by restriction digestion with restriction enzymes SwaI (methylation-insensitive) and EagI (methylation-sensitive). In this experiment, however, the digestion was performed either sequentially (FIG. 9A) or simultaneously (FIG. 9B). In both cases, methylation was assessed as either the presence or the absence of EagI cleavage sites as compared to the in silico restriction maps.

In sequential digests, the data is represented as the number of additional cuts per SwaI fragment. In simultaneous digests, EagI cuts appear as noise in the alignment of SwaI optical maps to the in silico restriction map.

### Example 4

### Using Fluorochrome-Labeled Binding Proteins as Methylation-Sensitive Reagents

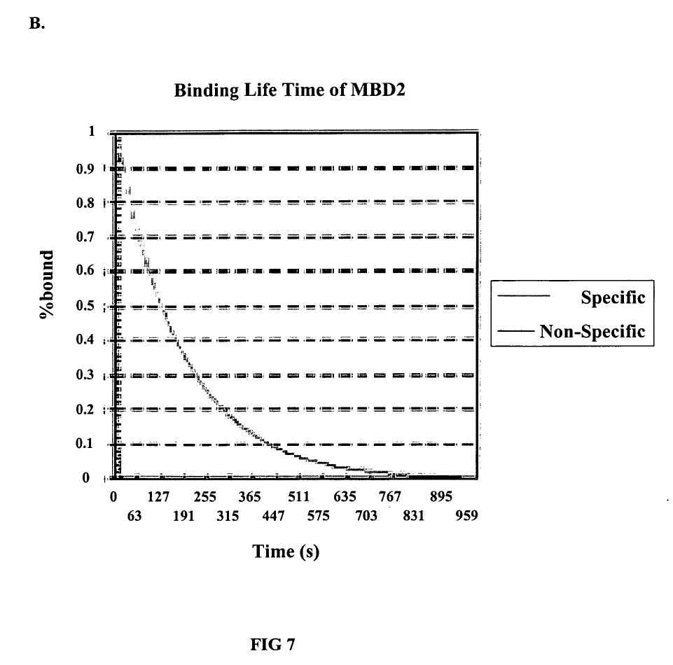

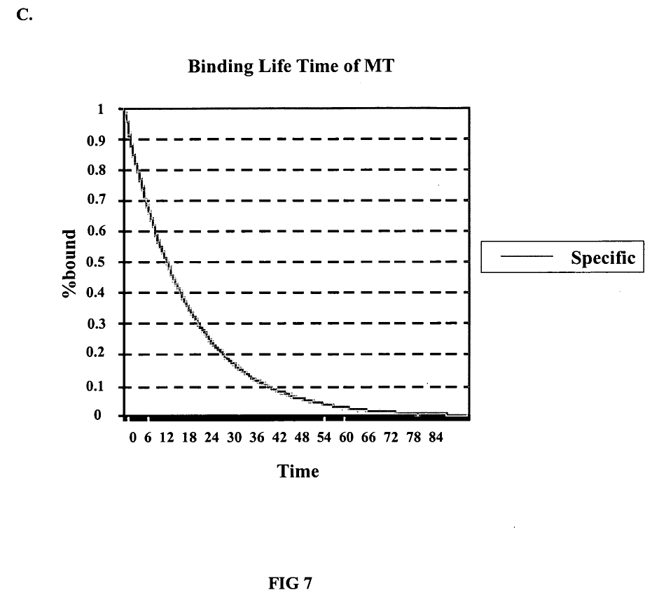

Polypeptides, such as methylation binding domains, can also be used as methylation-specific reagents. FIG. 7A shows the use of fragments of a binding protein having at least one or multiple fluorochrome-labeled methylation binding domains aligned to a bar code of an in silico digest of a genomic DNA molecule having methylation sites and a bar code of the genomic DNA molecule after restriction digestion and optical mapping, showing the sequence recognized by the methylation binding domain.

FIG. 7B shows a graph of DNA binding lifetime of MDBP2 protein. MDBP2 binds a single fully methylated CpG with a specific binding constant of K_{d}=2.7±0.08 nM. The constant for non-specific binding is K_{d}=188.7±46.8 nM. Assuming a diffusion association constant of Kₐ=2×10⁶ M/S, the half-life of specific interaction can be calculated to be 128.33 seconds, while the half-life of a non-specific interaction is 1.86 seconds. The high specificity of MDBP2 for its target and the relatively long half-life of the interaction make it possible to use MDBP2 as a probe of genomic methylation.

For example, the nucleic acid is indexed by digestion with a methylation-insensitive restriction enzyme such as SwaI. Labeled MDBP2 is bound to the DNA molecules by applying the protein dissolved in solution to the immobilized DNA molecules. Acetylated BSA is used as a blocker of non-specific binding of the labeled MDBP2 molecules. The surface is then washed for forty seconds. Following the wash, approximately 80% of specific sites and 1.9×10⁻⁷% of non-specific sites are occupied by MDBP2. The protein is non-reversibly cross-linked to the DNA, for example, by incubating the surface with a 1% formaldehyde solution in a non-Tris buffer at room temperature for 10 to 30 minutes. The immobilized nucleic acid is then imaged for YOYO-1 and for fluorochrome-labeled methylated DNA binding protein having excitation and emission spectra distinguishable from those of the nucleic acid dye. The imaging process is fully automated and proceeds across user-defined wavelength channels. The Channel Collect program merges individual images based on the CCD camera images. Since both the MDBP2 and YOYO-1 images are collected along the same set of defined coordinates, their precise alignment to each other is possible. The Peakfinder program is used to identify MDBP2 signals and to correlate them with the DNA bar codes. By correlating the MDBP2 signals with available sequence information, one can identify putative sites of CpG methylation. A suitable initial test genome is Lambda DNA fully methylated with the methylase SssI.

The CpG binding properties of labeled MLL protein or its MT domain can also be used in conjunction with optical mapping to characterize genome-wide methylation patterns in elongated, immobilized DNA molecules that are indexed by a restriction enzyme digest, especially hypomethylated regions and unmethylated CpG islands, as the MT polypeptide interacts with unmethylated CpG with a K_{d}=3.3×10⁻⁸ M. Assuming a Kₐ=2×10⁶ M/s, the half-life of the specific interaction is 10.5 seconds. FIG. 7C is a graph of the DNA binding lifetime of the MT domain. The data acquisition process is essentially the same as described above for the MDBP2 binding, but the binding of labeled MT generates a data set inverse to the MDBP2 data set. A drawback of MT is the short half-life of the specific binding interaction, which can be addressed by adapting harsher, but shorter washing conditions.

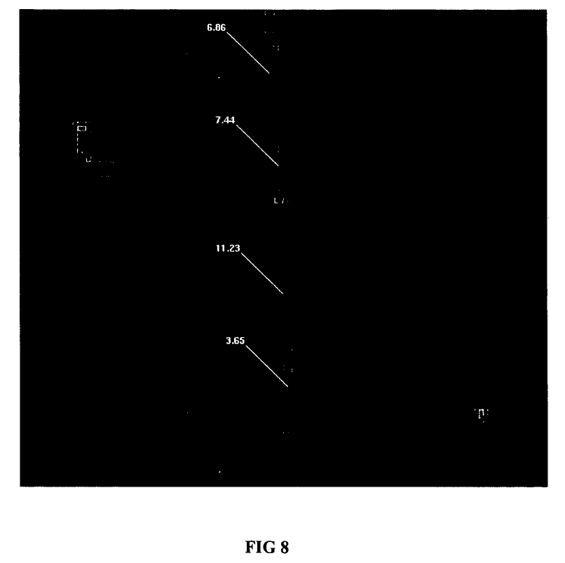

FIG. 8 shows an image of a single genomic DNA molecule labeled with YOYO-1 with superimposed circular indicators of the binding of the protein fragments labeled with a fluorochrome having excitation and emission spectra distinguishable from those of YOYO-1.

### Example 5

### Methylation Profiles of CpG Islands in Human Embryonic Stem Cell Genomic DNA

The methylation status of all 1256 CpG islands located in the genome of human embryonic stem cells (H1 line) was assessed using the double-digest approach. Single genomic DNA molecules were indexed by cleavage with the methylation-insensitive restriction enzyme SwaI, which generated an approximately 7× coverage of the human genome. A second restriction digest with methylation-sensitive restriction enzyme EagI was used to query the methylation status of specific loci, and produced about 1× coverage of the human genome.

Loci in the genome that were on a SwaI fragment and contained at least one CpG island that itself contained at least one EagI site were selected. EagI cleavage nicely targets CpG islands, since about 40% of the approximate 27,000 CpG islands in the human genome show at least one EagI site. Using these criteria, 625 sites of hypermethylation of CpG islands and 631 sites of hypomethylation were putatively identified.

Table 3 summarizes the distribution on each chromosome of CpG islands and EagI cleavage sites in the human genome. The average size of a CpG island is 763 base pairs and, on average, each CpG island is expected to have 2.2 EagI cleavage sites.

TABLE 3
Distribution of CpG Islands and EagI Sites on Human Chromosomes.

| | No. of CpG Islands | CpG Islands/MB | No. EagI Cuts |
|---|---|---|---|
| Chr1 | 2,416 | 10.85 | 5,186 |
| Chr2 | 1,669 | 7.03 | 4,445 |
| Chr3 | 1,155 | 5.94 | 3,172 |
| Chr4 | 1,011 | 5.40 | 2,687 |
| Chr5 | 1,227 | 6.90 | 2,985 |
| Chr6 | 1,257 | 7.51 | 2,935 |
| Chr7 | 1,528 | 9.89 | 3,561 |
| Chr8 | 1,030 | 7.22 | 2,648 |
| Chr9 | 1,208 | 10.28 | 2,912 |
| Chr10 | 1,149 | 8.74 | 2,920 |
| Chr11 | 1,369 | 10.44 | 3,089 |
| Chr12 | 1,221 | 0.93 | 2,763 |
| Chr13 | 605 | 6.33 | 1,497 |
| Chr14 | 787 | 8.91 | 1,881 |
| Chr15 | 786 | 9.67 | 1,960 |
| Chr16 | 1,485 | 18.82 | 3,026 |
| Chr17 | 1,623 | 20.86 | 3,324 |
| Chr18 | 507 | 6.79 | 1,360 |
| Chr19 | 2,492 | 44.66 | 3,689 |
| Chr20 | 800 | 13.47 | 1,856 |
| Chr21 | 355 | 10.38 | 920 |
| Chr22 | 714 | 20.52 | 1,741 |
| ChrX | 876 | 5.84 | 2,304 |
| ChrY | 172 | 7.05 | 362 |
| Total | 24,442 | 11.01797471 | |

Attention was focused initially on the methylation profile of DMBX1, a gene on human chromosome 1 that encodes a member of the bicoid subfamily of homeodomain-containing transcription factors that may play a role in brain and sensory organ development. Two transcript variants have been identified for this gene. The genomic sequence of the SwaI fragment associated with the DMBX1 gene and CpG islands contains 11 EagI cleavage sites, all of which are associated with CpG islands. The data from the SwaI/EagI double digests demonstrate that no EagI cuts are present in this SwaI fragment, indicating that all of the EagI sites are methylated. However, imaging of other SwaI restriction fragments of a different single genomic hES DNA molecule near the molecule containing the DMBX1 gene reveal additional cuts after EagI digestion, demonstrating a lack of methylation of EagI restriction sites known from the genomic sequence to be within the fragments.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

The invention claimed is:

1. A method for identifying a methylation profile in an immobilized, elongated polynucleotide, the method comprising the steps of:
cleaving the polynucleotide with a methylation-insensitive restriction enzyme;
imaging the cleaved polynucleotide;
further cleaving the imaged polynucleotide with a methylation-sensitive restriction enzyme;
imaging the further cleaved polynucleotide; and
comparing the images and intramolecular position to reference data to identify the methylation profile in the polynucleotide.

2. A method as recited in claim 1, wherein cleaving by the methylation-insensitive restriction enzyme and the methylation-sensitive enzyme are sequential.

3. A method as recited in claim 1, wherein cleaving by the methylation-insensitive restriction enzyme and the methylation-sensitive enzyme are simulataneous.

4. A method as recited in claim 1, wherein the polynucleotide is an isolated genomic DNA molecule.

5. A method as recited in claim 1, wherein the methylation-insensitive restriction enzyme is selected from the group consisting of BamHI, BisI and SwaI.

6. A method as recited in claim 1, wherein the methylation-sensitive restriction enzyme is selected from the group consisting of EagI, NheI, NotI, StuI and XhoI.

7. A method for identifying a methylation profile in an immobilized, elongated polynucleotide, the method comprising the steps of:
indexing the polynucleotide with respect to at least one restriction enzyme cleavage sequence;
selectively binding a labeled methylation-specific reagent to a site on the indexed polynucleotide; and
mapping the labeled reagent to the polynucleotide.

8. A method as recited in claim 7, wherein the methylation-specific reagent is a polypeptide.

9. A method as recited in claim 8, wherein the polypeptide comprises a methylation binding domain.

10. A method as recited in claim 9, wherein the polypeptide is selected from the group consisting of methylated DNA binding protein 1, methylated DNA binding protein 2, methylated DNA binding protein 3, methyl-CpG binding protein 1 and methyl-CpG binding protein 2.


SEQUENCE LISTING (1 sequence)

SEQ ID NO: 1 - DNA, 54 bp, Artificial
  Synthetic oligonucleotide
agctcgggcgtacggccgtatttttaaacccgcgcgcgggcggcgccgatatcg